(19) United States
(12) Patent Application Publication
(10) Pub. No.: US 2025/0108386 A1
(43) Pub. Date: Apr. 3, 2025

(54) Centrifuge chamber, cell centrifugation method and centrifugation device

(71) Applicant: SHENZHEN CELLBRI BIO-INNOVATION TECHNOLOGY CO., LTD, Shenzhen (CN)

(72) Inventors: Jinxin Zhang, Shenzhen (CN); Xiaoliang Guo, Shenzhen (CN); Yuanfang Shang, Shenzhen (CN)

(21) Appl. No.: 18/964,313

(22) Filed: Nov. 29, 2024

Related U.S. Application Data
(63) Continuation of application No. PCT/CN2023/109690, filed on Jul. 27, 2023.

(30) Foreign Application Priority Data
Jun. 1, 2022 (CN) 202210618036.0

(51) Int. Cl. B04B 1/04; B04B 7/12; C12M 1/26

(52) U.S. Cl. CPC B04B 1/04; B04B 7/12; C12M 33/10

(57) ABSTRACT

Provided are a centrifuge chamber, cell centrifugation method and centrifugation device. The centrifuge chamber includes a chamber bowl, chamber cover, first, second, and third area partition plates; a preset gap exists between the first area partition plate and an inner wall of the chamber bowl at an end of the side wall facing the chamber bowl; flow release ports are formed between the outer end of the second/third area partition plates and the inner wall; and the chamber cover and/or the chamber bowl are provided with liquid and liquid outlet passages; an liquid inlet passage port is positioned in the preset gap, and a distance from the inner wall is within a first preset range; a distance between an liquid outlet passage port and the inner wall is within a second preset range, and the liquid outlet passage port is positioned between the second and third area partition plates.

### CROSS REFERENCE TO RELATED APPLICATIONS

The present application is a Continuation Application of PCT Application No. PCT/CN2023/109690 filed on Jul. 27, 2023, which claims the benefit of Chinese Patent Application No. 202210618036.0 filed on Jun. 1, 2022. All the above are hereby incorporated by reference in their entirety.

### TECHNICAL FIELD

The application relates to the technical field of cell processing, in particular to a centrifuge chamber, a cell centrifuge method and a centrifugation device.

### BACKGROUND

In the field of cell processing, it is important to concentrate the cell fluid; that is, to convert low-concentration cell fluid into high-concentration cell fluid for further cell processing, such as storage and transportation. In view of this, centrifugal sedimentation method is commonly adopted to concentrate cells. The method includes: adding the cell fluid into the centrifuge chamber, the cells are pushed to the edge of the centrifuge chamber by centrifugal force at high rotation speed, and the supernatant near the rotation center of the centrifuge chamber is extracted to complete the cell fluid concentration. Two characteristic indexes, yield and viability, are critical for the centrifugal sedimentation method for cell concentration. The yield is a measure of the proportion of cells that leak from the centrifuge chamber outlet during concentration, i.e., Yield=the number of living cells after processed/the number of living cells before processed. The viability is a measure of the proportion of cells that survive fluid shear caused by centrifugation, i.e., Viability=the number of living cells/the total number of cells.

At present, when the rotating speed of most centrifuge chambers exceeds 3000 rpm, it causes significant shear damage to cells, resulting in low yield. When the rotating speed is less than 2500 rpm, the high flow rate cannot be employed, and the rotating speed and cell sedimentation speed are both reduced, resulting in a low activity rate. As a result, with the existing continuous flow concentration technology, the centrifuge chamber has a poor yield-activity rate and an unsatisfactory concentration effect at high flow rate and rotating speed.

### SUMMARY OF THE INVENTION

The application provides a centrifuge chamber, a cell centrifugation method and a centrifugation device. The centrifuge chamber can optimize the flow field based on continuous flow concentration, modify the cell trajectory, thus achieving less leakage and rapid concentration under the condition of high rotation speed and high flow rate, and enhancing both yield and viability.

In order to achieve the above purposes, the present application provides the following technical solutions.

The application provides a centrifuge chamber, which includes a chamber bowl, a chamber cover, a first area partition plate, a second area partition plate and a third area partition plate;
an extension direction of the first area partition plate is from a center of the chamber bowl to a side wall of the chamber bowl, and a preset gap exists between the first area partition plate and an inner wall of the chamber bowl at an end of the side wall facing the chamber bowl; flow release ports are formed between an outer end of the second area partition plate and the inner wall of the chamber bowl, and between an outer end of the third area partition plate and the inner wall of the chamber bowl; and
the chamber cover and/or the chamber bowl are provided with a liquid inlet passage and a liquid outlet passage; the liquid inlet passage port of the liquid inlet passage is positioned in the preset gap, and a distance between the liquid inlet passage port and the inner wall of the chamber bowl is within a first preset range; a distance between the liquid outlet passage port of the liquid outlet passage and the inner wall of the chamber bowl is within a second preset range, and the liquid outlet passage port is positioned between the second area partition plate and the third area partition plate.

Further, a preset gap exists between the first area partition plate and an inner wall of the chamber bowl at an end of the side wall facing the chamber bowl includes:
an end plate is arranged at the end of the side wall facing the chamber bowl, and a gap is formed between the end plate and the inner wall of the chamber bowl.

Further, the end plate is arc-shaped, with an arc length greater than or equal to 5 mm.

Further, the first preset range is 0.1 mm-20 mm.

Further, the second preset range is greater than or equal to 4 mm.

Further, the first area partition plate, the second area partition plate and the third area partition plate are sequentially arranged at intervals along a rotation direction of the chamber bowl; and
the first area partition plate, the second area partition plate and the third area partition plate all extend along a radial direction of the chamber bowl and are distributed in a Y shape; the first area partition plate is fixedly connected to the chamber cover or the chamber bowl provided with the liquid inlet passage, and the second area partition plate and the third area partition plate are fixedly connected to the chamber cover or the chamber bowl.

Furthermore, the flow release port formed between the outer end of the second area partition plate and the inner wall of the chamber bowl, and the flow release port formed between the outer end of the third area partition plate and the inner wall of the chamber bowl are both smaller than the distance between the liquid inlet passage port and the inner wall of the chamber bowl.

Further, the second area partition plate and the third area partition plate are distributed on two sides of the liquid outlet passage port.

In addition, the application provides a cell centrifugation method, which is performed to realize cell centrifugation using the centrifuge chamber described above, and the method includes:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

The application further provides a centrifugation device, which includes the centrifuge chamber described above.

Compared with the prior art, the application has the following beneficial effects.

1. The centrifuge chamber of the present application is provided with a first area partition plate, a second area partition plate and a third area partition plate, and the flow control of cell fluid is realized through three area partition plates. A preset gap is formed between the first area partition plate and an inner wall of the chamber bowl at an end of the side wall facing the chamber bowl, and a liquid inlet passage port for transporting cell fluid into the chamber bowl is arranged in the preset gap, so as to allow the position of the liquid inlet passage port for transporting cell fluid into the chamber bowl to be away from the rotation center, which provides a large centrifugal force for cell sedimentation. Meanwhile, the cell fluid enters the preset gap from the liquid inlet passage port, and the liquid flow direction changes to the tangential direction. Under this flow mode, the influence of fluid disturbance is less than that of centrifugal sedimentation, and the cells gradually adhere to the wall. As the cells carried by the liquid continue to flow along the circumferential wall, the fluid disturbance is further reduced and the cells fully settle to the wall. With the structural design of the preset gap and liquid inlet passage port, the liquid inlet speed and acceleration can be controlled, so as to control the cell trajectory, realize rapid cell sedimentation, and improve the yield and viability of centrifugation.

2. The centrifuge chamber of the present application is provided with three regional partition plates distributed in a Y shape, and an inlet area of cell fluid is formed between the arc-shaped end plate and the inner wall of the chamber bowl, and the inlet area is used to change the flow direction of the inlet fluid from radial to tangential. A development area is formed between the first area partition plate and the second area partition plate, and the development area is used to make the cell fluid continue to flow along the circumferential wall with the cells carried by the liquid, so that the fluid disturbance is further reduced and the cells fully settle to the wall. An outlet area is formed between the second area partition plate and the third area partition plate, and the outlet area is used to form a huge vortex to complete the separation of cells and supernatant, thus improving the yield and viability of centrifugation.

3. According to the centrifuge chamber of the present application, with the arrangement of the second area partition plate and the third area partition plate, flow release ports can be formed between the second area partition plate and the inner wall of the chamber bowl, and between the third area partition plate and the inner wall of the chamber bowl. The second area partition plate and the third area partition plate can provide a flow field similar to the preset gap. The gap formed by the flow release port guides the cells to move along the circumferential wall, forcing the cells to settle close to the circumferential wall, further avoiding cell disturbance, and keeping the cells in a state of adhering to the wall and continuously settling. Meanwhile, the second area partition plate and the third area partition plate are close to the liquid outlet passage port, when the cells adhere to the wall, the liquid flows out from the liquid outlet passage port, which prevents the cells from flying near the liquid outlet passage port, thus reducing the cell loss and improving the yield and viability of centrifugation.

4. According to the application, the liquid outlet passage port is arranged between the second area partition plate and the third area partition plate, the liquid forms a huge vortex between the second area partition plate and the third area partition plate, so that the cell-supernatant can be separated gently and clearly, and the yield and viability of centrifugation are improved.

Therefore, the centrifuge chamber with the above structure can optimize the cell movement in terms of the speed and acceleration of the liquid, reduce the cell leakage at low rotation speed and high flow rate, achieve rapid cell liquid concentration, and further enhance both the yield and viability.

### BRIEF DESCRIPTION OF THE DRAWINGS

FIG. 1 is an explosive view of the structure of the centrifuge chamber of the present application.

FIG. 2 is a sectional view of the centrifuge chamber of the present application.

FIG. 3 is a top view of the centrifuge chamber of the present application when the chamber cover is removed.

FIG. 4 is a flowchart of the cell centrifugation method of the present application.

Reference signs are as follows. 1—Chamber bowl; 2—Chamber cover; 3—First area partition plate; 4—Second area partition plate; 5—Third area partition plate; 6—End plate; 7—Preset gap; 8—First flow release port; 9—Second flow release port; 10—Liquid inlet passage; 11—Liquid outlet passage; 12—Liquid inlet port; 13—Liquid outlet port; 14—Liquid inlet passage port; 15—Liquid outlet passage port.

### DETAILED DESCRIPTION OF PREFERRED EMBODIMENTS

The technical solutions in the embodiments of the present application will be clearly and completely described below with reference to the drawings in the embodiments of this application. Obviously, the described embodiments are merely part of the embodiments of this application, not all of them. Based on the embodiments in this application, all other embodiments obtained by those of ordinary skill in the art without creative effort belong to the protection scope of this application.

### Embodiment 1

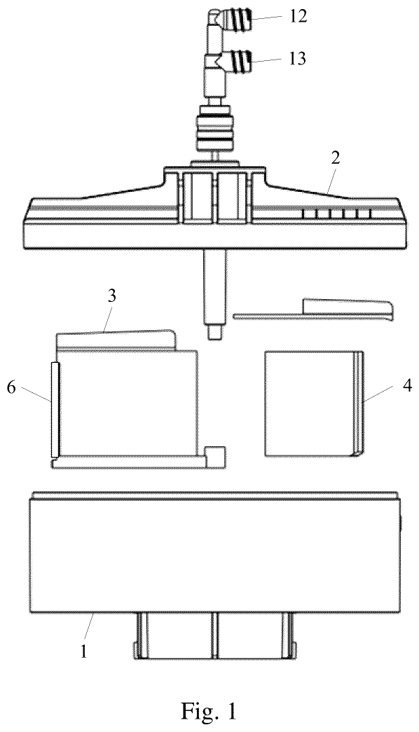

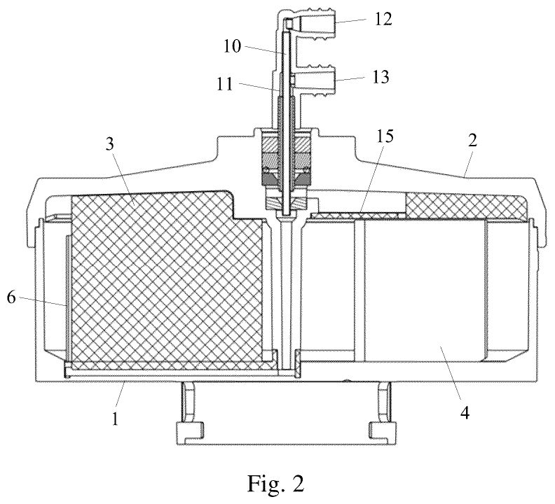

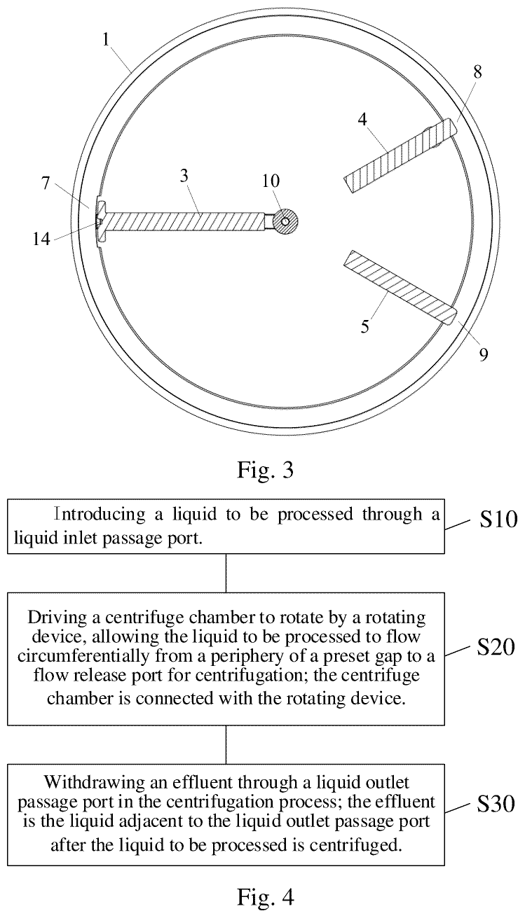

This embodiment provides a centrifuge chamber, as shown in the structures of FIG. 1, FIG. 2 and FIG. 3. The centrifuge chamber includes a chamber bowl 1, a chamber cover 2, a first area partition plate 3, a second area partition plate 4 and a third area partition plate 5. A circular cavity containing cell fluid is formed in the chamber bowl 1. The chamber cover 2 hermetically covers the top of the chamber bowl 1. The radius and height of the chamber bowl 1 can be designed according to requirements, and the radius of the chamber bowl 1 may be 30 mm-80 mm, the height of the chamber bowl 1 may also be 30 mm-80 mm, the radius of the chamber bowl 1 is preferably 30 mm, and the height of the chamber bowl 1 is preferably 35 mm.

An extension direction of the first area partition plate 3 is from a center of the chamber bowl 1 to a side wall of the chamber bowl 1, and a preset gap 7 exists between the first area partition plate 3 and an inner wall of the chamber bowl 1 at an end of the side wall facing the chamber bowl 1; flow release ports are formed between an outer end of the second area partition plate 4 (i.e., the end close to the inner wall of the chamber bowl 1) and the inner wall of the chamber bowl 1, and between an outer end of the third area partition plate 5 (i.e., the end close to the inner wall of the chamber bowl 1) and the inner wall of the chamber bowl 1.

The chamber cover 2 and/or the chamber bowl 1 are provided with a liquid inlet passage 10 and a liquid outlet passage 11; the liquid inlet passage port 14 of the liquid inlet passage 10 is positioned in the preset gap 7, and a distance between the liquid inlet passage port 14 and the inner wall of the chamber bowl 1 is within a first preset range; a distance between the liquid outlet passage port 15 of the liquid outlet passage 11 and the inner wall of the chamber bowl 1 is within a second preset range, and the liquid outlet passage port 15 is positioned between the second area partition plate 4 and the third area partition plate 5. Specifically, the first preset range and the second preset range are designed according to requirements, for example, the first preset range is 0.01 mm-30 mm, the second preset range is 4 mm-20 mm, and so on.

In one embodiment, the first preset range is 0.1 mm-20 mm.

In one embodiment, the second preset range is greater than or equal to 4 mm.

In an embodiment, when cell fluid needs to be concentrated by centrifuge chamber, the chamber bowl 1 rotates clockwise. As shown in FIG. 3, the first area partition plate 3, the second area partition plate 4 and the third area partition plate 5 are sequentially arranged at intervals along an rotation direction of the chamber bowl 1. The first area partition plate 3, the second area partition plate 4 and the third area partition plate 5 can also be arranged at intervals in sequence along the opposite rotation direction of the chamber bowl 1. The first area partition plate 3, the second area partition plate 4 and the third area partition plate 5 all extend along a radial direction of the chamber bowl 1 and are distributed in a Y shape. The first area partition plate 3 is fixedly connected to the chamber cover 2 or the chamber bowl 1 provided with the liquid inlet passage 10, and the second area partition plate 4 and the third area partition plate 5 are fixedly connected to the chamber cover 2 or the chamber bowl 1. There are gaps between the second area partition plate 4 and the first area partition plate 3, between the second area partition plate 4 and the third area partition plate 5, and between the third area partition plate 5 and the first area partition plate 3. The included angle between the second area partition plate 4 and the third area partition plate 5 is 10°˜120°, e.g., 10°, 20°, 30°, 40°, 50°, 60°, 80°, 90°, 100° and 120°; As shown in FIG. 3, preferably, the included angle between the second area partition plate 4 and the third area partition plate 5 is 60°.

The first area partition plate 3 extends from the center of the chamber bowl 1 toward the side wall of the chamber bowl 1, and a preset gap 7 exists between the first area partition plate 3 and an inner wall of the chamber bowl 1 at an end of the side wall facing the chamber bowl 1. In one embodiment, an end plate 6 is arranged at an end of the side wall facing the chamber bowl 1, and a gap is formed between the end plate 6 and the inner wall of the chamber bowl 1, which can help to reduce the damage to cells when the liquid inlet passage port 14 enters the liquid; that is, the gap forms preset gap 7. In an embodiment, an end plate 6 is arranged at an end of the side wall facing the chamber bowl 1. In an embodiment, the curvature of the end plate 6 may be the same as that of the inner side wall of the chamber bowl 1, thereby forming an arc-shaped gap between the end plate 6 and the inner wall of the chamber bowl 1. One end of the first area partition plate 3 is arranged near the center of the chamber bowl 1 and coincides with the rotation axis of the chamber bowl 1, and the other end is close to the inner side wall of the chamber bowl 1 and fixedly connected with the end plate 6/ The arc length of the end plate 6 is greater than or equal to 5 mm. A first flow release port 8 is formed between the outer end of the second area partition plate 4 and the inner wall of the chamber bowl; and a second flow release port 9 is formed between the outer end of the third area partition plate 5 and the inner wall of the chamber bowl 1. The distances between the end plate 6, the second area partition plate 4 and the third area partition plate 5 and the side wall of the chamber bowl 1 are all 0.1 mm-10 mm. The distance between the second area partition plate 4 and the inner wall of the chamber bowl 1 may be 3 mm. The distance between the third area partition plate 5 and the inner wall of the chamber bowl 1 may be 3 mm. The distance between the liquid inlet passage port 14 and the side wall of the chamber bowl 1 is smaller than that between the end plate 6 and the side wall of the chamber bowl 1.

The shape of the end plate 6 can be designed according to requirements, e.g. the shape of the end plate 6 is arc, T-shape, triangle and so on.

In an embodiment, the first flow release port 8 formed between the outer end of the second area partition plate 4 and the inner wall of the chamber bowl 1, and the second flow release port 9 formed between the outer end of the third area partition plate 5 and the inner wall of the chamber bowl 1 are both smaller than the distance between the liquid inlet passage port 14 and the inner wall of the chamber bowl 1. The chamber cover 2 and/or the chamber bowl 1 are provided with the liquid outlet passage 10 and the liquid outlet passage 11. That is, the liquid inlet passage 10 can be arranged on the chamber cover 2 or the chamber bowl 1, or both the chamber cover 2 and the chamber bowl 1. Similarly, the liquid outlet passage 11 can be arranged in the same way. As shown in FIG. 2 and FIG. 3, the liquid inlet passage 10 first extends to the inner bottom face of the chamber bowl 1 along the centers of the chamber cover 2 and the chamber bowl 1, and then continues to extend to the preset gap 7 along the bottom edge of the first area partition plate 3 to connect the liquid inlet port 12 and the liquid inlet passage port 14. The liquid outlet passage 11 extends along the radial direction of the rotation center and the bottom face of the chamber cover 2 to connect the liquid outlet port 13 and the liquid outlet passage port 15. The liquid inlet port 12 of the liquid inlet passage 10 and the liquid outlet port 13 of the liquid outlet passage 11 may both be positioned at the top of the chamber cover 2. The liquid inlet passage port 14 of the liquid inlet passage 10 is positioned in the preset gap 7, and the distance between the liquid inlet passage port 14 and the inner wall of the chamber bowl 1 is 5 mm. The liquid outlet passage port 15 of the liquid outlet passage 11 is arranged at the bottom of the chamber cover 2, and he distance between the liquid outlet passage port 15 and the inner wall of the chamber bowl 1 is 20 mm-30 mm; preferably 25 mm. The liquid outlet passage port 15 is positioned between the second area partition plate 4 and the third area partition plate 5. The second area partition plate 4 and the third area partition plate 5 are symmetrically distributed on the two sides of the liquid outlet passage port 15. The second area partition plate 4 and the third area partition plate 5 can likewise be distributed about symmetrically on the two sides of the liquid outlet passage port 15. The areas of the liquid inlet passage port 14 and the liquid outlet passage port 15 are both less than 9 mm², and the areas of the liquid inlet passage port 14 and the liquid outlet passage port 15 are preferably 4 mm², and both the liquid inlet passage port 14 and the liquid outlet passage port 15 are square ports.

The centrifuge chamber is provided with a first area partition plate 3, a second area partition plate 4 and a third area partition plate 5, and the flow control of cell fluid is realized through three area partition plates. An arc-shaped end plate 6 is arranged at one end of the side wall of the first area partition plate 3 facing the chamber bowl 1, and an arc-shaped gap is formed between the end plate 6 and the inner wall of the chamber bowl 1. The liquid inlet passage port 14 for transporting cell fluid into the chamber bowl 1 is arranged in an arc-shaped gap, and an inlet area of cell fluid is formed in the arc-shaped gap, so that the position of the liquid inlet passage port 14 for transporting cell fluid into the chamber bowl 1 is away from the rotation center. It provides a large centrifugal force for cell sedimentation, and meanwhile, the cell liquid enters the arc-shaped gap from the liquid inlet passage port 14, and is constrained by the gap wall. As a result, the liquid flow direction changes to the tangential direction, and the influence of fluid disturbance is less than that of centrifugal sedimentation, and the cells gradually adhere to the wall; as the cells carried by the liquid continue to flow along the circumferential wall, the fluid disturbance is further reduced and the cells fully settle to the wall. Through the structural design of the arc-shaped gap and liquid inlet passage port 14, the liquid inlet speed and acceleration can be controlled, so as to control the cell trajectory and realize the rapid cell sedimentation. With the arrangement of the end plate 6 at the end of the first area partition plate 3, the cell liquid flowing into the chamber bowl 1 can enter the arc-shaped gap from the liquid inlet passage port 14, and the cell liquid forms an annular liquid flow. At this state, the cell speed is along the circumferential direction, which is convenient for controlling the initial movement characteristics and initial trajectory of the cells, and controlling the initial trajectory of the cells is the key to the continuous sedimentation of the cells.

According to the centrifuge chamber of the present application, with the arrangement of the second area partition plate 4 and the third area partition plate 5, a flow release port 8 can be formed between the second area partition plate 4 and the inner wall of the chamber bowl 1, and a second flow release port 9 can be formed between the third area partition plate 5 and the inner wall of the chamber bowl 1. The second area partition plate 4 and the third area partition plate 5 can provide a flow field similar to the arc-shaped gap between the chamber bowl 1. The gap formed by the flow release port guides the cells to move along the circumferential wall face, forcing the cells to settle close to the circumferential wall, further avoiding cell disturbance, and enabling the cells to keep adhering to the wall surface and continuously settle. When the distance between the liquid outlet passage port 15 and the inner wall of the chamber bowl 1 is a second preset range, it is preferable that the second preset range is 20 mm-30 mm, so that the liquid is centrifuged to form an annular liquid level when the centrifuge chamber rotates. When the liquid is higher than the liquid outlet passage port 15, it will be quickly withdrawn, so as to stabilize the liquid level, ensure that the fluid trajectory is consistent and easy to control, and further ensure the liquid flow control effect of the first flow release port 8. Meanwhile, the second area partition plate 4 and the third area partition plate 5 are close to the liquid outlet passage port 15, and the liquid flows out from the liquid outlet passage port 15 when the cells adhere to the wall, thus preventing the cells from flying near the liquid outlet passage port 15, and further reducing the cell loss.

According to the centrifuge chamber of the present application, the liquid outlet passage port 15 is arranged at the bottom of the chamber cover 2, so that the liquid outlet passage port 15 is positioned at the top of the chamber bowl 1. With the arrangement of the liquid outlet passage port 15, the liquid reaching the liquid outlet passage port 15 can flow out of the centrifuge chamber, so that the liquid level position in the centrifuge chamber can be finalized and the liquid level in the centrifuge chamber can be kept stable.

The centrifuge chamber is provided with three area partition plates with Y-shaped distribution on the chamber cover 2 or the chamber bowl 1, and an inlet area of cell fluid is formed between the arc-shaped end plate 6 and the inner wall of the chamber bowl 1, and the inlet area is used for changing the inflow direction from radial to tangential. A development area is formed between the first area partition plate 3 and the second area partition plate 4, and the development area is used to make the cell liquid continue to flow along the circumferential wall with the cells carried by the liquid, allowing the fluid disturbance to be further reduced and the cells to fully settle to the wall. The effect of cell sedimentation can be further consolidated by following the flow tendency at the arc-shaped gap through the development area. With the arrangement of the liquid outlet passage port 15 between the second area partition plate 4 and the third area partition plate 5, an outlet area is formed between the second area partition plate 4 and the third area partition plate 5, and the outlet area is used to make the liquid form a huge vortex between the second area partition plate 4 and the third area partition plate 5, so that the cell-supernatant can be separated, and the yield and activity of centrifugation are improved.

Therefore, the centrifuge chamber with the above structure can optimize the cell movement in terms of the speed and acceleration of the liquid, reduce the linkage of cells at high speed and high flow rate, realize the rapid concentration of cell fluid, and further improve both the yield and viability.

Further, there are radial gaps between the second area partition plate 4 and the first area partition plate 3, and between the third area partition plate 5 and the first area partition plate 3.

Verification with T cells. The following are the yield and viability of human T cells concentrated by the aforementioned centrifuge chamber, as confirmed by many concentration experiments.

TABLE 1
Results of human t cell concentration test

| | | | | | Cell | |
|---|---|---|---|---|---|---|
| Serial | | Cell | Sample | Activity | number | |
| number | Sample | density | volume/ml | rate/% | *10⁶ | Yield/% |
| 1 | T cell | 2.35E+06 | 2086.00 | 92.35% | 4.90E+09 | / |
| | Yield | 6.60E+07 | 74.00 | 89.00% | 4.88E+09 | 99.63% |
| 2 | T cell | 2.32E+06 | 2142.00 | 97.65% | 4.97E+09 | / |
| | Yield | 6.80E+07 | 71.20 | 96.00% | 4.84E+09 | 97.43% |
| 3 | T cell | 2.54E+06 | 2309.00 | 96.09% | 5.86E+09 | / |
| | Yield | 4.68E+07 | 120.00 | 95.25% | 5.62E+09 | 95.76% |
| 4 | T cell | 1.57E+06 | 2555.00 | 97.29% | 4.01E+09 | / |
| | Yield | 4.90E+07 | 80.00 | 92.16% | 3.92E+09 | 97.72% |
| 5 | T cell | 1.09E+08 | 1539.00 | 96.90% | 1.68E+11 | / |
| | Yield | 9.40E+08 | 171.50 | 88.00% | 1.61E+11 | 96.10% |
| 6 | T cell | 5.75E+07 | 974.00 | 95.41% | 5.60E+10 | / |
| | Yield | 3.00E+08 | 178.00 | 87.92% | 5.34E+10 | 95.35% |
| 7 | T cell | 2.98E+07 | 1902.00 | 95.00% | 5.67E+10 | / |
| | Yield | 4.68E+08 | 116.00 | 83.00% | 5.43E+10 | 95.78% |
| 8 | T cell | 5.75E+07 | 867.70 | 97.60% | 4.99E+10 | / |
| | Yield | 2.90E+08 | 170.00 | 89.00% | 4.93E+10 | 98.81% |
| 9 | T cell | 3.80E+07 | 946.00 | 98.25% | 3.59E+10 | / |
| | Yield | 2.31E+08 | 152.00 | 95.00% | 3.51E+10 | 97.67% |
| 10 | T cell | 8.45E+07 | 1038.00 | 97.20% | 8.77E+10 | / |
| | Yield | 3.20E+08 | 267.30 | 94.00% | 8.55E+10 | 97.52% |
| 11 | T cell | 4.27E+07 | 1469.00 | 96.15% | 6.27E+10 | / |
| | Yield | 3.53E+08 | 170.00 | 90.20% | 6.00E+10 | 95.65% |
| 12 | T cell | 2.56E+08 | 1340.00 | 96.79% | 3.43E+11 | / |
| | Yield | 1.22E+09 | 272.20 | 92.37% | 3.32E+11 | 96.81% |
| 13 | T cell | 8.45E+07 | 1038.00 | 97.20% | 8.77E+10 | / |
| | Yield | 3.20E+08 | 267.30 | 94.00% | 8.55E+10 | 97.52% |
| 14 | T cell | 1.12E+08 | 2538.00 | 97.94% | 2.84E+11 | / |
| | Yield | 6.32E+08 | 436.00 | 92.00% | 2.76E+11 | 96.94% |
| 15 | T cell | 2.14E+08 | 1462.00 | 93.88% | 3.13E+11 | / |
| | Yield | 6.00E+08 | 516.00 | 92.00% | 3.10E+11 | 98.96% |
| 16 | T cell | 1.53E+08 | 2135.00 | 98.00% | 3.27E+11 | / |
| | Yield | 5.70E+08 | 565.00 | 90.40% | 3.22E+11 | 98.59% |
| 17 | T cell | 2.03E+06 | 5614.00 | 88.43% | 1.14E+10 | / |
| | Yield | 4.50E+07 | 242.20 | 87.54% | 1.09E+10 | 95.61% |
| 18 | T cell | 2.56E+06 | 2152.00 | 95.31% | 5.51E+09 | / |
| | Yield | 1.16E+08 | 47.00 | 94.17% | 5.43E+09 | 98.54% |
| 19 | T cell | 1.51E+06 | 2330.00 | 96.28% | 3.52E+09 | / |
| | Yield | 2.80E+07 | 120.50 | 93.66% | 3.37E+09 | 95.90% |
| 20 | T cell | 1.95E+08 | 2514.00 | 86.20% | 4.90E+11 | / |
| | Yield | 9.19E+08 | 517.70 | 70.83% | 4.76E+11 | 97.10% |
| 21 | T cell | 1.70E+06 | 2405.00 | 94.31% | 4.09E+09 | / |
| | Yield | 1.36E+07 | 294.40 | 92.97% | 4.00E+09 | 97.93% |

By adopting the centrifuge chamber, continuous flow concentration of cell fluid can be realized, and a continuous flow can be formed between the liquid inlet port and the liquid outlet port. The flow rate of cell fluid can reach 100 ml/min-250 ml/min, and the rotating speed of the centrifuge chamber can be 2000 rpm˜2500 rpm during the concentration process.

When the liquid outlet passage port 15 of the centrifuge chamber is arranged at the bottom of the chamber cover 2, the liquid outlet passage port 15 is located at the top of the chamber bowl 1. With the arrangement of the liquid outlet passage port 15, the liquid reaching the liquid outlet passage port 15 can flow out of the centrifuge chamber, so that the liquid level position in the centrifuge chamber can be finalized, and the liquid level in the centrifuge chamber can be kept stable.

### Embodiment 2

This embodiment provides a cell centrifugation method. As shown in FIG. 4, the cell centrifugation method performs cell centrifugation through the centrifuge chamber of the above embodiment, and the cell centrifugation method includes the following steps.

Step S10: introducing a liquid to be processed through a liquid inlet passage port 14.

Step S20: driving a centrifuge chamber to rotate by a rotating device, allowing the liquid to be processed to flow circumferentially from a periphery of a preset gap 7 to a flow release port for centrifugation; the centrifuge chamber is connected with the rotating device.

S30: withdrawing an effluent through a liquid outlet passage port 15 in the centrifugation process; the effluent is the liquid adjacent to the liquid outlet passage port 15 after the liquid to be processed is centrifuged.

The liquid to be processed is a solution requiring cell centrifugation, and the liquid to be processed flows in from the liquid inlet passage port 14, so that an inlet area of cell liquid is formed between the arc-shaped end plate 6 and the inner wall of the chamber bowl 1, and the inlet area is used for changing the inflow direction from radial to tangential. A development area is formed between the first area partition plate 3 and the second area partition plate 4, and the development area is used to make the cell fluid continue to flow along the circumferential wall with the cells carried by the liquid, allowing the fluid disturbance to be further reduced and the cells to fully settle to the wall. During the rotation of the centrifuge chamber, the effect of cell sedimentation can be further consolidated by following the flow tendency at the preset gap 7 through the development area. With the arrangement of the liquid outlet passage port 15 between the second area partition plate 4 and the third area partition plate 5, an outlet area is formed between the second area partition plate 4 and the third area partition plate 5, and the outlet area is used to make the liquid form a huge vortex between the second area partition plate 4 and the third area partition plate 5. As a result, the application realizes that the liquid to be processed flows in through the liquid inlet passage port 14; the centrifuge chamber is driven to rotate by the rotating device, so that the liquid to be processed flows from the periphery of preset gap 7 to the liquid release port for centrifugation. The centrifuge chamber is connected with the rotating device. In the process of centrifugation, the effluent is withdrawn through the liquid outlet passage port 15, so that the separation between cells and supernatant can be gently and clearly completed, thus withdrawing the effluent, which is the liquid adjacent to the liquid outlet passage port 15 after the liquid to be processed is centrifuged, thereby improving the yield and activity of the effluent obtained by centrifugation.

### Embodiment 3

This embodiment provides a centrifugation device, which includes the centrifuge chamber described in the above embodiment, and performs cell centrifugation through the centrifuge chamber, which is an operation process of concentrating cells by centrifugal sedimentation.

The centrifugation device adopts the centrifuge chamber of the present application, thus continuous flow concentration of cell fluid can be realized, and the yield and viability of cell fluid during centrifugation can be improved.

Apparently, those skilled in the art can make various modifications and variations to the embodiments of the present application without departing from the spirit and scope of the present application. Therefore, the present application is intended to encompass these modifications and variations as long as they fall within the scope of the claims and their equivalents.

1. A centrifuge chamber, comprising a chamber bowl, a chamber cover, a first area partition plate, a second area partition plate, and a third area partition plate;
an extension direction of the first area partition plate is from a center of the chamber bowl to a side wall of the chamber bowl, and a preset gap exists between the first area partition plate and an inner wall of the chamber bowl at an end of the side wall facing the chamber bowl; flow release ports are formed between an outer end of the second area partition plate and the inner wall of the chamber bowl, and between an outer end of the third area partition plate and the inner wall of the chamber bowl; and
the chamber cover and/or the chamber bowl are provided with a liquid inlet passage and a liquid outlet passage; an liquid inlet passage port of the liquid inlet passage is positioned in the preset gap, and a distance between the liquid inlet passage port and the inner wall of the chamber bowl is within a first preset range; a distance between an liquid outlet passage port of the liquid outlet passage and the inner wall of the chamber bowl is within a second preset range, and the liquid outlet passage port is positioned between the second area partition plate and the third area partition plate.

2. The centrifuge chamber of claim 1, wherein a preset gap exists between the first area partition plate and an inner wall of the chamber bowl at an end of the side wall facing the chamber bowl comprises:
an end plate is arranged at the end of the side wall facing the chamber bowl, and a gap is formed between the end plate and the inner wall of the chamber bowl.

3. The centrifuge chamber of claim 2, wherein the end plate is arc-shaped, with an arc length greater than or equal to 5 mm.

4. The centrifuge chamber of claim 1, wherein the first preset range is 0.1 mm-20 mm.

5. The centrifuge chamber of claim 1, wherein the second preset range is greater than or equal to 4 mm.

6. The centrifuge chamber of claim 1, wherein the first area partition plate, the second area partition plate and the third area partition plate are sequentially arranged at intervals along a rotation direction of the chamber bowl; and
the first area partition plate, the second area partition plate and the third area partition plate all extend along a radial direction of the chamber bowl and are distributed in a Y shape; the first area partition plate is fixedly connected to the chamber cover or the chamber bowl provided with the liquid inlet passage, and the second area partition plate and the third area partition plate are fixedly connected to the chamber cover or the chamber bowl.

7. The centrifuge chamber of claim 1, wherein the flow release port formed between the outer end of the second area partition plate and the inner wall of the chamber bowl, and the flow release port formed between the outer end of the third area partition plate and the inner wall of the chamber bowl are both smaller than the distance between the liquid inlet passage port and the inner wall of the chamber bowl.

8. The centrifuge chamber of claim 6, wherein the second area partition plate and the third area partition plate are distributed on two sides of the liquid outlet passage port.

9. A cell centrifugation method, wherein the cell centrifugation method is performed to realize cell centrifugation using the centrifuge chamber of claim 1, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

10. A centrifugation device, comprising the centrifuge chamber of claim 1.

11. A cell centrifugation method, wherein the cell centrifugation method is performed to realize cell centrifugation using the centrifuge chamber of claim 2, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

12. A cell centrifugation method, wherein the cell centrifugation method is performed to realize cell centrifugation using the centrifuge chamber of claim 4, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

13. A cell centrifugation method, wherein the cell centrifugation method is performed to realize cell centrifugation using the centrifuge chamber of claim 5, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

14. A cell centrifugation method, wherein the cell centrifugation method is performed to realize cell centrifugation using the centrifuge chamber of claim 6, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

15. A cell centrifugation method, wherein the cell centrifugation method is performed to realize cell centrifugation using the centrifuge chamber of claim 7, and the method comprises:
introducing a liquid to be processed through a liquid inlet passage port;
driving a centrifuge chamber to rotate by a rotating device, allowing the liquid to be processed to flow circumferentially from a periphery of a preset gap to a flow release port for centrifugation; the centrifuge chamber is connected with the rotating device; and
withdrawing an effluent through a liquid outlet passage port in the centrifugation process; the effluent is the liquid adjacent to the liquid outlet passage port after the liquid to be processed is centrifuged.

16. A centrifugation device, comprising the centrifuge chamber of claim 2.

17. A centrifugation device, comprising the centrifuge chamber of claim 4.

18. A centrifugation device, comprising the centrifuge chamber of claim 5.

19. A centrifugation device, comprising the centrifuge chamber of claim 6.

20. A centrifugation device, comprising the centrifuge chamber of claim 7.
